(19) United States
(12) Patent Application Publication
(10) Pub. No.: US 2022/0061699 A1
(43) Pub. Date: Mar. 3, 2022

(54) Flexible Capacitive Sensing Mat Including Spacer Fabric

(71) Applicant: Apple Inc., Cupertino, CA (US)

(72) Inventors: Daniel W. LaBove, San Francisco, CA (US); Chin San Han, Mountain View, CA (US); Ali M. Amin, Cupertino, CA (US); Henry Rimminen, Espoo (FI); Riley E. Brandt, Menlo Park, CA (US); Timothy L. Weadon, Mountain View, CA (US); Yindar Chuo, San Jose, CA (US); Zijing Zeng, San Jose, CA (US)

(21) Appl. No.: 17/008,240

(22) Filed: Aug. 31, 2020

(51) Int. Cl. A61B 5/11; G01L 1/14; A61B 5/00; A47C 21/00

(52) U.S. Cl. CPC A61B 5/1102; G01L 1/146; A61B 5/6892; A61B 2562/0247; A61B 2562/182; A61B 2562/164; A61B 2562/028; A47C 21/00

(57) ABSTRACT

Disclosed herein are capacitive sensors, and methods for their operation, that determine the presence of a user by detecting input forces and/or pressures. Capacitive sensors may be in the form of a flexible capacitive sensing mat. An electronic device incorporating a flexible capacitive sensing mat may include spacer fabric disposed between conductive layers. The spacer fabric may have a first thickness and may be configured to compress to at least a second thickness when a threshold pressure is applied to the layered sensor and revert to the first thickness when the threshold pressure is no longer applied to the flexible mat. A capacitive sense circuit electrically coupled to the second conductive layer and configured to generate a presence detection signal when the spacer fabric layer compresses to the second thickness may additionally be provided.

### FIELD

Embodiments described herein generally relate to capacitive sensors that may be used to detect a presence, a pressure, or an input force and, in particular, to flexible sensing mats including a layer made of a spacer fabric.

### BACKGROUND

Sleep deficiency is a large and growing problem in modern society. Under guidelines from the United States' Centers for Disease Control and Prevention (CDC), for example, adults need 7 or more hours of sleep per night for optimal health. Adults sleeping less than the recommended 7 hours typically display a higher rate of health-related complications such as: worsening symptoms from chronic diseases (e.g., asthma); increased chance of chronic conditions such as heart attacks, heart disease, and stroke; increased chance of mental disorders such as depression; and worsening performance in school- or work-related tasks.

Treatment for sleep deficiency generally includes medication, cognitive treatments, and/or behavioral counseling. For example, meditation techniques may be performed by an individual before sleep in an attempt to relax and experience better quality rapid eye movement (REM) sleep. In another example, medication may depress the central nervous system and may cause an individual to more easily fall asleep.

These treatments, however, do not directly measure and monitor an individual's sleep. When attempting to diagnose problems related to sleep deficiency, therefore, medical and/or individuals may generally rely on estimations about an individual's sleep patterns. Such estimations may include errors due to the difficulty in reliably obtaining and tracking such information.

### SUMMARY

A flexible mat may comprise a first conductive layer, an electromagnetic shield, a second conductive layer disposed between the first conductive layer and the electromagnetic shield, and a spacer fabric disposed between the first conductive layer and the second conductive layer. The spacer fabric may have a first thickness and may be configured to compress to at least a second thickness when an input is applied to the flexible mat, the second thickness less than the first thickness, and revert to the first thickness when the input is no longer applied to the flexible mat. The flexible mat may additionally comprise a capacitive sense circuit electrically coupled to the second conductive layer and configured to generate a presence detection signal when the spacer fabric layer compresses to the second thickness.

In some embodiments, the spacer fabric may comprise a first fabric layer, a second fabric layer, and a synthetic monofilament layer disposed between the first fabric layer and the second fabric layer. The first fabric layer and the second fabric layer may be formed from at least one of a spandex material, a polyethylene terephthalate material, a cotton, or a wool. The synthetic monofilament layer may have a compressive modulus between 10 kilopascals (kPa) and 20 kPa. In some embodiments, the synthetic monofilament layer may have a cross-shaped pattern.

In some embodiments, a threshold pressure may be 1.5 kPa and the capacitive sense circuit may generate the presence detection signal when the input applies the threshold pressure, or any pressure greater than the threshold pressure, to the flexible mat.

The first conductive layer may be bonded to the spacer fabric by a first adhesive layer and the second conductive layer may be bonded to the spacer fabric by a second adhesive layer. In some embodiments, a line stitch may be disposed at an end of the flexible mat and may be configured to couple the first conductive layer, the second conductive layer, and the spacer fabric.

An electronic device may comprise a flexible housing defining an interior volume and a flexible sensing strip positioned within the interior volume. The flexible sensing strip may comprise a first conductive layer, a second conductive layer, and a spacer fabric disposed between the first conductive layer and the second conductive layer, the spacer fabric configured to deform in response to an input. The electronic device may further comprise a capacitive sense circuit electrically coupled to the flexible sensing strip and configured to generate a detection signal when the flexible sensing strip deforms in response to the input. The capacitive sense circuit may additionally enable an operation of a biometric sensor once the detection signal is generated.

In some embodiments, the first conductive layer may be a first conductive thread, the second conductive layer may be a second conductive thread, the first conductive thread may be woven into a first surface of the spacer fabric, and the second conductive thread may be woven into a second surface of the spacer fabric, the second surface opposite the first surface.

In some embodiments, a biometric sensor may be at least one of the flexible sensing strip, a ballistocardiograph sensor, a piezoelectric sensor, a heart-tracking monitor, or a micro-electromechanical system device.

In some embodiments, the first conductive layer and the second conductive layer may define a plurality of electrode pairs and each electrode pair of the plurality of electrode pairs may comprise a first electrode in the first conductive layer and a second electrode in the second conductive layer. The capacitive sense circuit may generate the detection signal when a distance between the upper electrode and the lower electrode of any one of the plurality of electrode pairs satisfies a threshold.

In some embodiments, the flexible sensing strip may extend across a width of a mattress and the input may correspond to a presence of a user on the mattress. The spacer fabric may have a first gap material modulus lower than a second gap material modulus of the mattress. In some embodiments, the flexible sensing strip may be aligned with a chest of the user while the user lies on the mattress.

A flexible mat may comprise a first conductive layer, a second conductive layer, and a spacer fabric disposed between the first conductive layer and the second conductive layer. The spacer fabric may comprise a first fabric layer, a second fabric layer, and a synthetic monofilament layer disposed between the first fabric layer and the second fabric layer.

The flexible mat may further comprise an electromagnetic shield configured to prevent electromagnetic signals from interfering with the first conductive layer and the second conductive layer. The second conductive layer may be disposed between the electromagnetic shield and the first conductive layer.

In some embodiments, the synthetic monofilament layer may have a compressive modulus between 10 kPa and 20 kPa. The synthetic monofilament layer may have an angle, with respect to the first conductive layer, greater than or equal to 45 degrees. A thickness of the flexible mat when fully compressed may be between 0.3 mm and 3 mm.

### BRIEF DESCRIPTION OF THE DRAWINGS

Reference will now be made to representative embodiments illustrated in the accompanying figures. It should be understood that the following descriptions are not intended to limit the embodiments to one or more preferred embodiments. To the contrary, they are intended to cover alternatives, modifications, and equivalents as may be included within the spirit and scope of the described embodiments as defined by the appended claims. Identical reference numerals have been used, where possible, to designate identical features that are common to the figures.

FIG. 1A illustrates a top view of an example flexible mat with associated electronics, as described herein.

FIG. 1B illustrates a cross-sectional view of an example flexible mat including a flexible sensor strip, as described herein.

FIG. 1C illustrates a cross-sectional view of an example flexible mat including a flexible sensor strip when an input is applied to the flexible mat, as described herein.

FIG. 2 illustrates an example flexible sensor strip including conductive layers bonded to a spacer fabric by an adhesive, as described herein.

FIG. 3 illustrates an example flexible sensor strip including a line stitch coupling a first conductive layer, a second conductive layer, and a spacer fabric, as described herein.

FIG. 4 illustrates an example flexible sensor strip with conductive layers made of threads of conductive filament, as described herein.

FIG. 5A illustrates a front view of a user lying on a mattress with an example above-mattress flexible mat, as described herein.

FIG. 5B illustrates a side view of a user lying on a mattress with an example above-mattress flexible mat, as described herein.

FIG. 6A illustrates an example flexible sensor strip including a monofilament spacer fabric disposed with an angle with respect to a first conductive layer, as described herein.

FIG. 6B illustrates the flexible sensor strip including the monofilament spacer fabric of FIG. 6A when an input is applied to the flexible sensor strip, as described herein.

FIG. 7 illustrates an example flexible sensor strip including a monofilament layer with a cross-shaped pattern, as described herein.

FIG. 8A illustrates an example flexible sensor strip including a number of conductive pixels, as described herein.

FIG. 8B illustrates the flexible sensor strip of FIG. 8A when an input is applied to a portion thereof, as described herein.

FIG. 9 depicts a flowchart of an example process for tuning spacer fabric properties, as described herein.

The use of cross-hatching or shading in the accompanying figures is generally provided to clarify the boundaries between adjacent elements and to facilitate legibility of the figures. Accordingly, neither the presence nor the absence of cross-hatching or shading conveys or indicates any preference or requirement for particular materials, material properties, element proportions, element dimensions, commonalities of similarly illustrated elements, or any other characteristic, attribute, or property for any element illustrated in the accompanying figures.

Additionally, it should be understood that the proportions and dimensions (either relative or absolute) of the various features and elements (and collections and groupings thereof), and the boundaries, separations, and positional relationships presented therebetween, are provided in the accompanying figures merely to facilitate an understanding of the various embodiments described herein, may not necessarily be presented or illustrated to scale, and are not intended to indicate any preference or requirement for an illustrated embodiment to the exclusion of embodiments described with reference thereto.

### DETAILED DESCRIPTION

The following disclosure relates to health monitoring devices for detecting the presence of a person or object on a mattress or cushioned furniture. In particular, a health monitoring device may be a flexible mat with a first conductive layer, a second conductive layer, and a compliant layer disposed in between the first and second conductive layers. The compliant layer may be formed from a spacer fabric with particular properties. Example spacer fabric properties may include: spacer fabric material (e.g., monofilament or multifilament); monofilament or multifilament density; monofilament or multifilament angle; knitting machine parameters; material treatments; compressive modulus (e.g., Young's modulus) values; maximum or minimum compressive thickness; and so on. In some embodiments, a health monitoring device may be placed on a mattress or cushioned furniture and may be configured to detect the presence of a user sitting, lying, and/or otherwise positioned on the mattress or cushioned furniture.

In some embodiments, a health monitoring device may be configured to be imperceptible to a user while the user is sitting, lying, and/or otherwise positioned on the mattress or cushioned furniture. In this way, the comfort of the mattress and/or cushioned furniture may be unaffected by the health monitoring device.

In various embodiments discussed herein, a health monitoring device may be a flexible mat and may include a number of different sensing components such as biometric sensors. The flexible mat may additionally be configured to extend across a width of a mattress or cushioned furniture. The flexible mat may include a number of different sensing components and/or biometric sensors including: a capacitive sensor; a ballistocardiograph sensor; a micro-electro-mechanical system (MEMS); a touch sensor; any combination thereof; and so on. A flexible mat may be affixed to a mattress or cushioned furniture by, for example, an adhesive or fastener (e.g., hook and loop fastener), and may operate as a health monitoring device to detect certain vital signs (e.g., body weight, body temperature, pulse rate, respiration rate, blood flow, and so on). A flexible mat may additionally detect sleep-related metrics such as snoring, the amount of tossing and turning undergone by a user, and so on.

To prevent a flexible mat from attempting to measure a user's vital signs when the user is not present, a detection sensor (e.g., a presence detection sensor or presence sensor) may be provided. The detection sensor may detect the user's presence on the mattress or cushioned furniture and may activate or inactivate associated sensors (e.g., a biometric sensor) of the flexible mat based on the detection of the user's presence. In some embodiments, the detection sensor may operate as a biometric sensor and may, in addition to detecting a user's presence, measure biometric signals such as a respiration rate, a heart rate, and so on.

In some embodiments, a capacitive sensor may operate as a detection sensor. A capacitive sensor may be referred to as a flexible sensor strip and may be integrated into a flexible mat, such as described herein. The flexible mat may include a flexible housing and may include first and second conductive layers separated by a compliant layer positioned within the flexible housing. A capacitance value between the first and second conductive layers may be detected by a capacitive sense circuit. When an input is applied to the flexible mat, the first and second conductive layers may move toward each other, resulting in a changed or changing capacitance value and a deformation of the compliant layer. A threshold capacitance, corresponding to a particular distance between the first and second conductive layers, may be stored in a capacitive sense circuit and an input may be detected once the threshold capacitance is met or surpassed. When the input is removed from the flexible mat, the compliant layer may operate as a spring and restore the default separation between the first and second conductive layers. In some embodiments, a magnitude of an input may be determined by measuring a change in the capacitance value corresponding to the input. The threshold capacitance value may correspond to a detected pressure imparted on the flexible mat.

Due to the use of the flexible mat in an on-bed or on-furniture environment, the flexible mat desirably comprises materials that are nearly imperceptible to a user when in use while still providing consistent presence detection. Since mattresses and cushioned furniture are typically designed for comfort, a rigid mat disposed on top of the mattress or cushioned furniture may be undesirable (e.g., uncomfortable). However, some flexible mats formed partially of, for example, foam may exhibit poor resiliency and/or presence detection capabilities. As discussed throughout this disclosure, a spacer fabric layer may be provided between conductive layers of a flexible mat to rectify both of these concerns. While a spacer fabric is described in this disclosure, the material is not limited as such. In some embodiments, a foam or a buckling silicone material may be provided between conductive layers of a flexible mat.

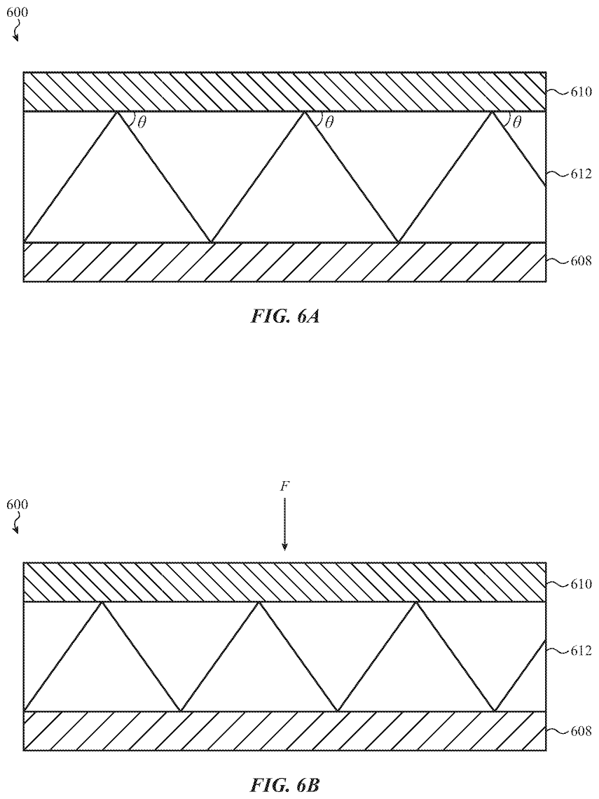

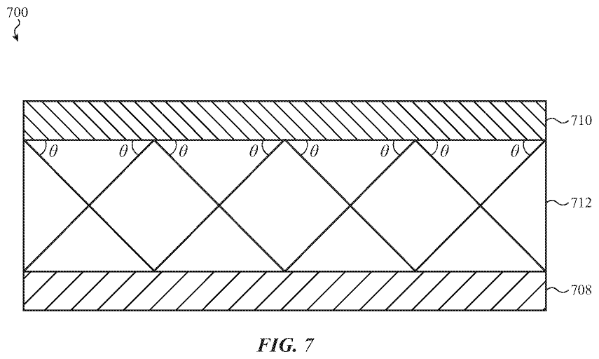

As used herein, a spacer fabric is a three dimensional textile structure that is constructed, for example, from yarns, polymers, fabric, monofilaments, multifilaments, and so on. Examples of spacer fabric construction are shown in FIGS. 6A, 6B, and 7. As illustrated, spacer fabrics may be disposed between a top surface and a bottom surface of a mat and may control an equilibrium distance between the top surface and the bottom surface. In response to an applied input, the spacer fabric may temporarily deform, but may revert to the equilibrium distance once the input is removed.

A flexible mat including a spacer fabric layer may provide consistent presence detection even at low measured pressures (e.g., 1.5 kilopascals (kPa)), may tolerate multiple cycles of compression and restoration, and may be largely imperceptible to a user when installed on a mattress or cushioned furniture. The spacer fabric layer may exhibit low hysteresis and high resiliency due to low, or no, viscoelastic behavior. In this way, consistent presence detection may be performed without sacrificing a user's physical comfort.

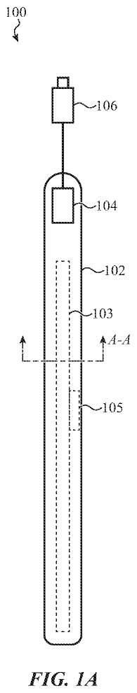

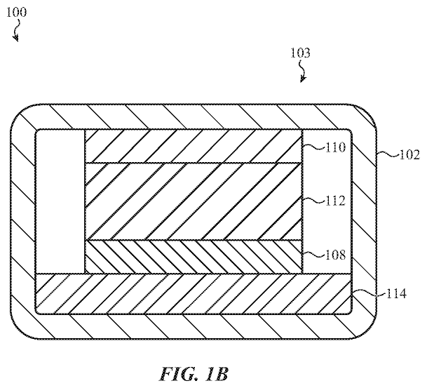

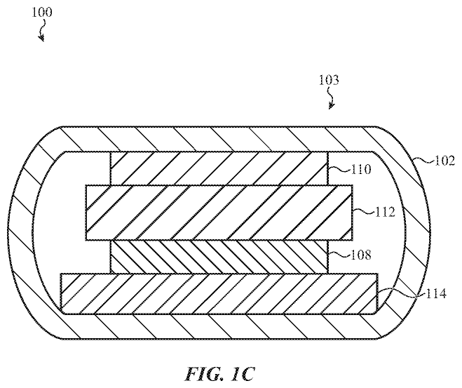

These and other embodiments are discussed with reference to FIGS. 1A-9. However, those skilled in the art will readily appreciate that the detailed description given herein with respect to these figures is for explanatory purposes only and should not be construed as limiting.

FIG. 1A illustrates a top view of an example flexible mat 100 that may be used to perform presence detection when positioned on top of a mattress or cushioned furniture. The flexible mat 100 may include features such as a flexible housing 102, flexible sensing strip 103, a capacitive sense circuit 104, a biometric sensor 105, and a power circuit 106. The flexible mat 100 may also include one or more internal components typical of an electronic device such as, for example, one or more processors, memory components, network interfaces, and so on. In addition to a flexible sensing strip 103, the flexible mat 100 may include a number of different sensors, circuits, and/or monitors such as a heart tracking monitor, a ballistocardiograph sensor, a piezoelectric sensor, a micro-electromechanical system (MEMS) device, any combination thereof, any associated circuitry, and so on.

For example, in FIG. 1A a flexible sensing strip 103 may be provided across a partial length of the flexible mat 100. The flexible sensing strip 103 may be a capacitive sensing strip and may include two capacitive layers moveable with respect to one another. In response to an input on the flexible mat 100, the capacitive layers may move closer to one another. As discussed herein, a threshold capacitance value may be established and a detection signal (e.g., a presence detection signal) may be generated when the threshold capacitance value is met or surpassed. A biometric sensor 105 may be provided proximate to the flexible sensing strip 103. The biometric sensor 105 may be configured to detect a user's biometrics. The biometric sensor 105 may be turned on or activated once a presence detection signal is generated in response to the flexible sensing strip 103 detecting an input sufficient for the generation of a detection signal. In some embodiments, the biometric sensor 105 may be positioned underneath an area where a user's heart will most likely align. In some embodiments, the biometric sensor 105 may be a strip or may comprise a number of sensors. In some embodiments, before a detection signal is generated, the biometric sensor 105 may be disabled. Once the presence detection signal is generated in response to the flexible sensing strip 103 detecting an input, the biometric sensor 105 may be enabled.

In a non-limiting example, the biometric sensor 105 may be a ballistocardiograph sensor and may measure ballistic forces generated by a user's heart, for example, as a user's heart pumps blood through the user's veins. Information captured via the biometric sensor 105 may be used to detect a user's cardiovascular health and may be used in a number of diagnostic tools.

The flexible sensing strip 103 may, in some embodiments, operate as an additional biometric sensor and may detect biometric and/or physiological signals from a user. For example, the flexible sensing strip 103 may detect a user's respiration rate; heart rate; signals representative of bodily sounds (e.g., a cough, a heart murmur, or a stomach rumbling); and so on. The flexible sensing strip 103 may detect, by capacitive measurements, any motion (e.g., a motion of a user's body) that cause mechanical distortion in the flexible sensing strip 103. As a non-limiting example, motion of a user's torso due to respiration may cause pressure modulation at an interface between the user and the flexible sensing strip 103. This pressure modulation may then modulate the flexible sensing strip 103 and result in a measured capacitance. This capacitance may be used to derive, for example, a respiration rate and an inspiration-exhalation (I-E) ratio. Similarly, the contraction of the heart may cause a blood flow that results in an inertial motion of the body. This inertial movement of the body may be captured as a ballistocardiographic (BCG) signal as detected from capacitive signals from the flexible sensing strip 103. A similar mechanism for sensor distortion may occur for other body motions that may result in capacitive signals that correlate to biometric and/or physiological signals, such as seismocardiographic movements; muscle movements (e.g., tossing, turning, or restlessness; and acoustic vibrations (e.g., coughing or chest, heart, or stomach sounds). The flexible sensing strip 103 may additionally capture static pressure signals, which can potentially be used to derive the weight of a user on the sensor and/or a change in the user's weight over time.

In some embodiments, the biometric sensor 105 may be omitted and the flexible sensing strip 103 may operate as the sole biometric sensor. Additionally, the flexible sensing strip 103 may comprise multiple modes such as a full-operation mode and a limited sensing (low-power state) mode. In a non-limiting example, a full-operation mode may allow the flexible sensing strip 103 to capture any number of presence and/or biometric signals. A limited sensing mode may disable some biometric capture features while still permitting presence sensing to occur. In some embodiments, a limiting sensing mode may transition into a full operation mode once a user's presence is detected and for a period of time afterwards.

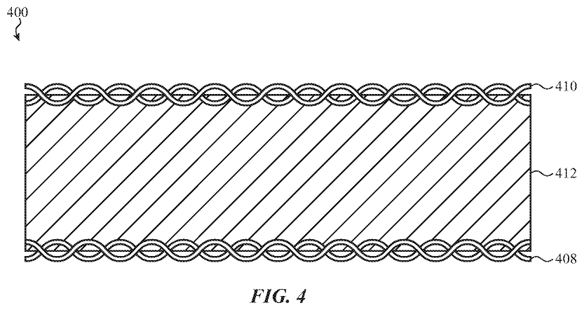

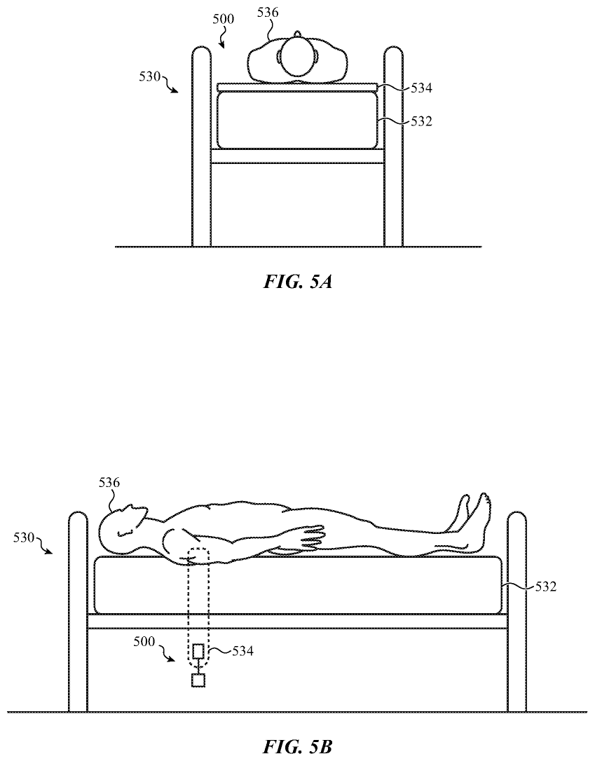

In the example depicted in FIG. 1A, the flexible mat 100 is shaped as a long strip. In some embodiments, the flexible mat 100 may have a width less than 1 centimeter and may have a length less than 1 meter, though other dimensions are considered as well. As discussed with respect to FIGS. 5A and 5B, the flexible mat 100 may be placed along a width of, for example, a mattress and may be aligned with a user's chest while the user is in a standard sleeping position. In some embodiments, the flexible mat 100 may be positioned across a width of a mattress and may be placed one-thirds down a length of the mattress with respect to a front end of the mattress. In non-depicted embodiments, the flexible mat 100 may have any shape, for example a circular- or square-shaped mat.

In the illustrated embodiment, the flexible housing 102 may be provided to protect internal components of the flexible mat 100. The flexible housing 102 may be formed from thin plastics such as polyethylene, polycarbonate, acrylic, polypropylene, and so on. In some embodiments, the flexible housing 102 may be formed from a fabric such as a spacer fabric, a natural fabric, a synthetic fabric, or any blend thereof, or may be formed from any other flexible material such as rubber (e.g., natural or synthetic rubber). In some embodiments, the flexible housing 102 may be completely eliminated such that a flexible sensing strip 103 is open to an external environment.

The capacitive sense circuit 104 may be coupled to a flexible sensing strip 103 and may be disposed within an internal cavity of the flexible housing 102 (see, e.g., FIGS. 1B and 1C). The flexible sensing strip 103 may be used to detect a presence of a user and a detection signal may be generated by the capacitive sense circuit 104. The detection signal may correspond to a capacitance value between two electrodes of the flexible capacitive sensing mat and the capacitive sense circuit 104 may include hardware and/or software components for establishing a threshold capacitance value. The threshold capacitance value may correspond to an input pressure/force applied to the flexible mat 100. In some embodiments, the operational parameters of the capacitive sense circuit 104 may be controlled by an external electronic device operatively coupled to the flexible mat 100 (e.g., by wireless or wired signals).

In some embodiments, the flexible housing 102 may define a mattress cover (e.g., a sheet) and may be integrated into the mattress cover. For example, the flexible housing may be a fitted or un-fitted sheet and may be placed over the flexible sensing strip 103 when a user changes bedsheets.

A power circuit 106 may be provided at one end of the flexible mat 100 and may be operatively coupled to internal components of the flexible mat 100 such as the capacitive sense circuit 104 and/or the flexible sensing strip 103. The power circuit 106 may couple the flexible mat 100 to an external power source via, for example, a universal serial bus (USB) adaptor or a 4-, 3-, or 2-pronged plug. In some embodiments, a replaceable and/or rechargeable battery may be coupled to the flexible mat 100. In some embodiments, the power circuit 106 may be configured to receive power from a charging element, such as a magnetic puck which may include an inductive coil and wireless charging elements.

FIG. 1B illustrates a cross-sectional view of the flexible mat 100 along the line A-A depicted in FIG. 1A. The flexible mat 100 may include the flexible sensing strip 103 which may include a first conductive layer 110, a second conductive layer 108, a spacer fabric 112, and an electromagnetic shield 114. A capacitive sense circuit may use the flexible sensing strip 103 to detect an input by detecting a capacitance between the first conductive layer 110 and the second conductive layer 108. In particular, an input may deform or deflect the flexible housing 102, thereby compressing or otherwise deforming the spacer fabric 112 and reducing a distance between the first conductive layer 110 and the second conductive layer 108. A threshold capacitance value may be established such that whenever the threshold capacitance value is met or exceeded, a detection signal may be generated. As discussed herein, the detection signal may indicate when a user is lying or sitting on a mattress or cushioned furniture.

In some embodiments, the second conductive layer 108 may be a sensing electrode and the first conductive layer 110 may be a grounded electrode. As depicted in FIG. 1C, the first conductive layer 110 may be moveable, with respect to the second conductive layer 108, in response to in input. In this way, a changing capacitance value may be measured between the second conductive layer 108 and the first conductive layer 110 using self-capacitance principles. In some embodiments, an alternating current (AC) signal may be applied across the first conductive layer 110 and a change in an associated electric field, in response to an input, may be detected (e.g., mutual capacitance). In some embodiments, a processor or circuit (e.g., the capacitive sense circuit) associated with the flexible sensing strip 103 may be configured to detect a threshold capacitance value corresponding to a presence of a user. The threshold capacitance value may correspond to a distance between the first conductive layer 110 and the second conductive layer 108 and may correspond to an input. The input may be an input force and may be measured as a pressure since the force acts over an area of the flexible mat 100.

The threshold capacitance value may correspond to a threshold pressure measured by the flexible mat 100. For example, an input may apply a force over an area of the flexible mat 100 and the capacitive sense circuit may detect a pressure from the applied force and capacitance value. In some embodiments, the threshold pressure may correspond to a pressure value of about 1.5 kPa. As used herein, about 1.5 kPa may refer to a value of 1.5 kPa+/−10%.

Noise from other electronic components of the flexible mat 100 and external electronic devices may result in electrical interference with the flexible sensing strip 103 and/or the capacitive sense circuit. In order to prevent electric field interference, an electromagnetic shield 114 may be provided between the second conductive layer 108 and the housing 102 to prevent the second conductive layer 108 from measuring an external capacitance that may result in a false positive presence detection signal. The electromagnetic shield 114 may be biased to a certain voltage (e.g., an active electromagnetic shield). If biased to a certain voltage, the electromagnetic shield 114 may be used to cancel interfering electric fields. In some embodiments, multiple electromagnetic shields may be provided. For example, an additional electromagnetic shield may be provided to be in contact with the first conductive layer 110.

In some embodiments, a pressure value may be detected by the flexible mat 100 instead of or in addition to presence detection. The deflection and/or compressive behavior of the spacer fabric 112 may be modeled so that a processor or circuit (e.g., the capacitive sense circuit) associated with the flexible mat 100 may determine an amount of pressure generated by an input force over an area of the flexible mat 100 for a given input. In particular, known forces may be applied to the flexible housing 102 in various locations to determine the change in capacitance resulting from a given amount of force applied to a given location. These forces may be converted to pressure values by components of the flexible mat 100 as an area of the flexible sensing strip 103 may be known. This information may be stored in a table, graph, as an equation representing a pressure versus capacitance curve, or in any other data structure or algorithm that may be used to correlate a capacitance value with a force value.

FIG. 1C illustrates a cross-sectional view of the flexible mat 100, along the line A-A depicted in FIG. 1A, as a force F is being applied to the flexible housing 102. As the force F is applied to the flexible housing 102, the spacer fabric 112 may compress and the first conductive layer 110 and the second conductive layer 108 may move towards each other. Once the spacer fabric 112 compresses by a certain amount, the threshold capacitance value may be met or surpassed and the capacitive sense circuit may generate a detection signal signifying that a certain pressure threshold has been met or surpassed. In some embodiments, the threshold capacitance value may correspond to a threshold pressure of about 1.5 kPa (e.g., a force F applied over an area of the flexible mat 100 results in a measured pressure) applied to the flexible housing 102. For the purposes of this description, about 1.5 kPa may refer to 1.5 kPa+/−10%.

Once the force F is removed, the spacer fabric 112 may act as a spring and return the flexible mat 100 to its original, uncompressed state (e.g., as depicted in FIG. 1B). As the spacer fabric 112 reverts to its original state, the spacer fabric 112 may force the distance between the first conductive layer 110 and the second conductive layer 108 to increase.

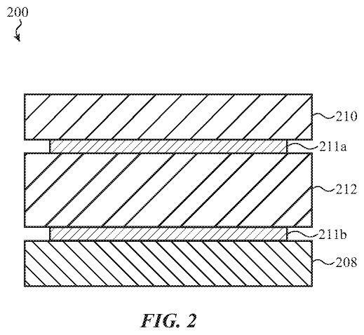

FIG. 2 illustrates a flexible sensing strip 200 including conductive layers bonded to a spacer fabric 212 by adhesive layers 211a and 211b. A first adhesive layer 211a may be disposed between the first conductive layer 210 and the spacer fabric 212 and a second adhesive layer 211b may be disposed between the second conductive layer 208 and the spacer fabric 212. The conductive layers may be formed fully or partially of a conductive material.

Each adhesive layer may be formed of a laminate to bond each conductive layer to respective ends of the spacer fabric 212 and may provide high longevity and resiliency while possessing elastic properties. Example laminate materials include thermoset adhesive materials, heat-activated films, polyurethane films, various polymers, water-absorbing materials, curing components, epoxy, acrylic, polyurethane, any combination thereof, and so on. By bonding the conductive layers to the spacer fabric 112 in this way, a strong bond between the conductive layers and the spacer fabric 112 may be formed. The spacer fabric 112 may move in conjunction with the conductive layers and may compress and revert with precision based on applied inputs.

FIG. 3 illustrates a flexible sensing strip 300 including conductive layers bonded to a spacer fabric 312 by line stitches 313a and 313b. A first line stitch 313a may be provided on one end of the flexible sensing strip 300 and may stitch a first conductive layer 310, the spacer fabric 312, and a second conductive layer 308 together. An opposite end of the flexible sensing strip 300 may be provided with a second line stitch 313b to stitch the first conductive layer 310, the spacer fabric 312, and the second conductive layer 308 together. Each line stitch may be made from a non-conductive thread so as to not interfere with capacitance values between the first conductive layer 310 and the second conductive layer 308.

The first conductive layer 310 and the second conductive layer 308 may each be formed fully or partially of a conductive material. In additional or alternative embodiments, the first conductive layer 310 and the second conductive layer 308 may be made of conductive thread, either entirely or a portion thereof, and may each be formed as a fabric layer on opposing surfaces of the spacer fabric 312. Though the line stitches 313a and 313b are depicted as a running stitch, any type of stitch may be used, including basting stitches, slip stitches, cross-stitches, backstitches, and so on. In some embodiments, a single stitch running through the center or along a width of the flexible sensing strip 300 may be used instead of or in addition to two stitches along either end of the flexible sensing strip 300.

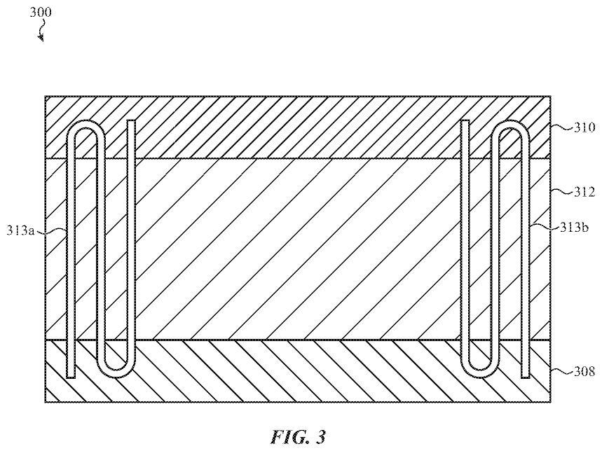

FIG. 4 illustrates a flexible sensing strip 400 including a first conductive thread layer 410 and a second conductive thread layer 408. In the depicted example, each of the first and second conductive thread layers may include electrical wiring, textile wiring (e.g., wiring surrounded by a fabric covering), and/or conductive thread. The first and second conductive thread layers may be woven into a top and bottom surface, respectively, of a spacer fabric 412.

Each of the first and second conductive thread layer may include a monofilament, or may include a braided structure as depicted in FIG. 4. In some embodiments, the first and second conductive thread layers may be comprised entirely of conductive thread along an entire surface of the spacer fabric 412. In other embodiments, the first and second conductive thread layers may be partially formed of conductive thread (e.g., along a length or width of the spacer fabric 412) in order to form a number of conductive pixels or in order to form a length-wise or width-wise conductive strip. In some embodiments, an electromagnetic shield may also be formed of a conductive thread and one or multiple electromagnetic shields may be woven into the second conductive layer 408 and/or the first conductive layer 410.

In some embodiments, the conductive thread layers may be formed from metal wires with an enamel coating. In other embodiments, the conductive thread layers may be formed from metal wires without an enamel coating. The materials used for the metal wires may be copper, silver-plated copper, brass, silver-plated brass, silver, stainless steel, any alloy thereof, and so on. In some embodiments, the diameter of each metal wire may be less than 1 mm, and in some cases may be between 0.02 mm and 0.5 mm. In alternative embodiments, the diameter of each metal wire may be greater than 1 mm.

As discussed herein, the conductive layers described with respect to FIG. 1A-4 may be formed from plaited or printed fabrics, printed electrodes, flex materials on substrates, woven conductive wires (see FIG. 4), and so on. As used herein, plaited fabrics may refer to a knit construction where a second type of yarn or thread is knit under a first type of yarn or thread. In a plaited construction, for example, one type of yarn or thread may include a conductive thread or other conductive material and another type of yarn or thread may be a non-conductive thread or material. A printed fabric may include a textile base with conductive ink printed onto the textile base to form a conductive layer. In such embodiments, the conductive ink may be formed in various patterns and may comprise one or any number of pixels. Printed electrodes may include, for example, screen-printed electrodes with polyethylene terephthalate substrates and carbon electrodes. A printed electrode may be formed in any shape and may include an adhesive to stick to a spacer fabric layer. Conductive layers may also partially or fully comprise a substrate with conductive traces.

As discussed herein, the spacer fabrics may be formed from any spacer fabric construction, such as via a warp knit or a circular knit. In a warp knit, a yarn or thread may be looped around an entire length of the fabric along adjacent columns and may form a loose knit. A circular knit may utilize a circular knitting jig and may refer to a knitting process where yarn or thread is knit around a circumference of a textile.

In some embodiments, the spacer fabric may instead be formed of a buckling silicone membrane. A buckling silicone membrane may be formed of silicone rubber and may include two relative stable states. For example, a first stable state may be when no force is applied to the buckling silicone membrane. A second stable state may be when a threshold force is applied to the buckling silicone membrane. For example, when no force is applied to the buckling silicone membrane after a force is applied, the buckling silicone membrane may transition from the second stable state to the first stable state. A buckling silicone membrane may be formed with any number of geometrical configurations and may have one or a number of collapsible zones.

In some embodiments, the spacer fabric may be formed as a composite stack-up including different cores having distinct thicknesses or elastic properties. For example, a spacer fabric may include three spacer fabric layers each comprising different stitching, fabrics, and/or densities. A spacer fabric layer adjacent to conductive layers may be relatively less elastic and an internal core may be relatively more elastic. A stack-up in this manner may increase a longevity of a flexible mat. In some embodiments, spacer fabric layers may surround buckling silicone membrane layers. Any combination of spacer fabric and/or buckling silicone membrane layers may be used in accordance with the provided disclosure.

FIGS. 5A and 5B illustrate an example of an operation for a flexible mat 500 when positioned on a mattress 532 and underneath a user 536. FIG. 5A depicts a front view of this example and FIG. 5B depicts a side view.

In the depicted example, a user 536 may lie on a mattress 532 with a flexible mat 500 positioned in between the mattress 532 and the user 536. In some embodiments, the flexible mat 500 may be operably connected to an external power supply (via, for example, a wall outlet) or may be powered by an internal or external battery.

As the user 536 lies on the flexible mat 500, the flexible mat 500 may be fully or partially compressed due to the weight of the user 536. As the flexible mat device 500 compresses, a flexible sensing strip within the flexible mat 500 may also compress (see, e.g., FIG. 1C). As the flexible sensing strip compresses, conductive layers of the flexible sensing strip may move closer together. Once these conductive layers are a certain distance apart (e.g., a distance corresponding to a threshold capacitance), a detection signal may be generated by electronics of the flexible mat 500 and the user's 536 presence on the mattress 532 may be detected. In some embodiments, the presence detection signal may be generated when a pressure of 1.5 kPa or greater (e.g., an input force detected over an area of the flexible mat) is imparted on the flexible mat 500.

In the example depicted in FIGS. 5A and 5B, a bed 530 includes a mattress 532 and a frame. A flexible mat 500 and a user 536 are positioned on a top surface of the mattress 532. In some embodiments, the flexible mat 500 (and spacer fabric of the flexible mat 500) may have a gap material modulus lower than the mattress 532. In this way, the health monitoring device 500 may be sufficiently flexible and nearly imperceptible to the user 536.

In some embodiments, the flexible mat 500 may be aligned with a user's 536 chest so as to detect a user's 536 heartbeat. In some embodiments, the flexible mat 500 may be positioned along any length- or width-wise direction with respect to the mattress 532.

Properties of the spacer fabric 612 will now be discussed with particular reference to FIGS. 6A and 6B. FIG. 6A illustrates a cross-section of an example flexible sensing strip 600 including a first conductive layer 610, a second conductive layer 608, and the spacer fabric 612. The first and second conductive layers may be any type of conductive layer as discussed herein and with respect to FIGS. 1A-5B. The first and second conductive layers may include a blend of materials including spandex/elastane, polyethylene terephthalate, conductive materials, cotton, wool, and so on.

The spacer fabric 612 may include a monofilament layer disposed between two fabric layers defining a first surface and a second surface. The monofilament layer may be three dimensionally sewn between the two fabric layers. The fabric layers may be formed from a blend of materials including spandex/elastane, polyethylene terephthalate, cotton, wool, and so on. In some embodiments, the fabric layers defining the first and the second surface may be formed of a material different from the monofilament layer. In some embodiments, a density of a monofilament in the monofilament layer may correspond to between about 5 denier and 50 denier. In some embodiments, a density of a monofilament in the monofilament layer may correspond to about 10 denier. As used herein, a denier refers to a measure of linear mass density for monofilaments in the monofilament layer and is the mass in grams of the monofilament per 9,000 meters of the monofilament. As provided herein, any dimensional value may be approximate values and may be +/−10% of the values disclosed herein.

The spacer fabric 612 may be a monofilament layer and may be positioned at a non-perpendicular angle (e.g., an angle θ) with respect to the first conductive layer 610. The monofilament layer may be sewn into the first and second conductive layers and the angle θ may remain relatively consistent even as the flexible sensing strip 600 compresses and reverts. In some embodiments, the angle θ may be between 90 degrees and 45 degrees. In some embodiments, the angle θ may be approximately 45 degrees. The monofilament layer may have a repeating pattern along the length of the flexible sensing strip 600 and each angle θ may be substantially equivalent.

In FIG. 6B, a force F is applied to a top surface of the first conductive layer 610 and compresses the flexible sensing strip 600. As the flexible sensing strip 600 is compressed, the angle θ changes as the monofilament layer compresses and buckles. The change of the angle θ results in energy stored within the monofilament layer. The stored energy is released once the force F is removed and the monofilament layer returns to its original thickness.

In some embodiments, a thickness of the flexible sensing strip 600 may have a limit when the flexible sensing strip 600 is fully compressed. For example, a pressure of, in a non-limiting example, 5 kPa applied to the spacer fabric 612 may fully compress the spacer fabric 612 so that pressures larger than 5 kPa do not additionally compress the flexible sensing strip 600.

In some embodiments, a thickness of the flexible capacitive sensing mat when fully compressed may be between 0.3 mm and 3 mm. In some embodiments, a thickness when fully compressed may be approximately 1.5 mm. Even after fully compressed as depicted in FIG. 6B, the spacer fabric 612 may fully revert to its shape depicted in FIG. 6A, since the spacer fabric 612 exhibits high resiliency and low or no viscoelastic behavior. In this way, successive capacitive measurements may be consistent across numerous force events. The compressive modulus of the spacer fabric 612 may be between 5 kPa and 100 kPa. In some embodiments, the compressive modulus of the spacer fabric 612 may be between 10 kPa and 20 kPa. As provided herein, any dimensional or pressure value may be approximate values and may be +/−10% of the values disclosed herein.

The monofilament spacer fabric 612 may comprise a synthetic, natural, or blended material such as polyester, nylon, cotton, any combination thereof, and so on. In some embodiments, the spacer fabric 612 may comprise a multifilament fabric formed from a number of different fabric strands. The fabric comprising the spacer fabric 612 may be heat-set and/or dyed. For example, the spacer fabric 612 may be flat scoured and/or rapid scoured in order to remove impurities, oils, and/or dirt that may have formed during a manufacturing process. The spacer fabric 612 may be dyed so as to give the flexible mat a desired aesthetic appearance. In some embodiments, the heat-set and/or dying processes may affect a compressive property of the spacer fabric 612 by, for example, weakening or strengthening physical properties of the spacer fabric 612 thread.

FIG. 7 illustrates a cross-section of an example flexible sensing strip 700 with a spacer fabric 712 having a cross-shaped pattern. The spacer fabric 712 may be sewn between a first conductive layer 710 and a second conductive layer 708.

In some embodiments, spacer fabric 712 may include a number of strands that cross or overlap between first and second conductive layers. As depicted in FIG. 7, the spacer fabric 712 may have an angle θ with respect to the first conductive layer 710. As described with respect to the example depicted in FIGS. 6A and 6B, the angle θ may be between 90 degrees and 45 degrees. In some embodiments, the angle θ may be approximately 45 degrees. The spacer fabric 712 may have a repeating pattern along the length of the flexible sensing strip 700 and each angle θ may be substantially equivalent.

In some embodiments, the cross-shaped spacer fabric 712 may be a monofilament spacer fabric and may be configured to cross over previously knit strands to form the cross shaped pattern depicted. The spacer fabric 712 may comprise a synthetic, natural, or blended material such as polyester, nylon, cotton, any combination thereof, and so on. In some embodiments, the spacer fabric 712 may comprise a multifilament fabric formed from a number of different fabric strands. The fabric comprising the spacer fabric 612 may be heat-set and/or dyed, as described above.

In some embodiments, the different rows of yarn or thread in a cross-knit pattern may be formed at different angles with respect to the first conductive layer 710. For example, one row of yarn or other spacer material may be oriented at an angle of 75 degrees with respect to the conductive layers 710, 712, and another row of yarn or other spacer material may be oriented at an angle of 45 degrees with respect to the conductive layers 710, 712. The provided values are merely explanatory and any value may be used in accordance with the provided disclosure.

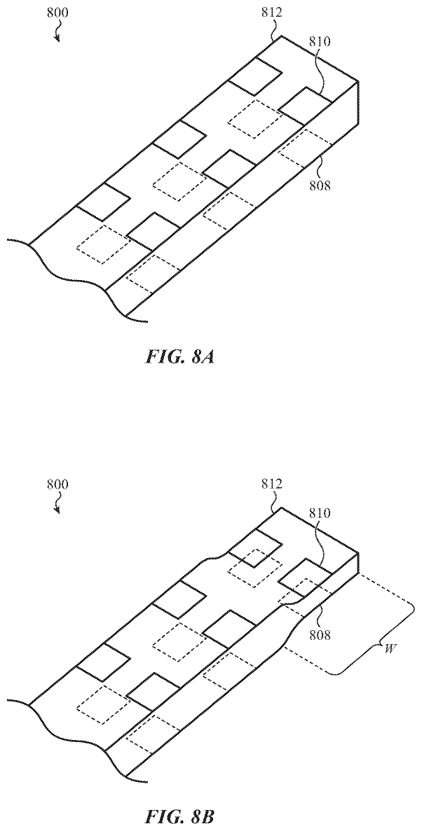

FIGS. 8A and 8B illustrate an example where a flexible sensing strip 800 is provided with multiple pixels defined by a first conductive pixel 810 and a second conductive pixel 808 (e.g., an electrode pair). A spacer fabric 812 may be formed as an extended layer and a number of conductive pixels may be interspersed throughout the spacer fabric 812. The conductive pixels may be formed as any conductive pixel including those discussed herein, such as laminate layer conductors, conductive fabrics, and so on. Though FIGS. 8A and 8B depict each conductive pixel as a uniform shape arranged in a regular pattern, any spacing, whether regular or irregular, any shape, whether uniform or non-uniform, and any size, whether uniform or non-uniform, may be provided. In some embodiments, a substrate may be provided on a top and/or bottom surface of the spacer fabric 812.

In some embodiments, a first conductive layer and a second conductive layer may be disposed on either side of the spacer fabric 812. The first conductive layer and the second conductive layer may comprise a number of conductive pixels (e.g., electrode pairs) disposed intermittently throughout the first and second conductive layer. Individual sensing circuits may be provided and may detect individual capacitance values between each set of conductive pixels, with each sensing circuit detecting the capacitance between one conductive pixel. In alternate or additional embodiments, a shared sense circuit may be provided to detect a capacitance value between each conductive pixels individually. As depicted in FIGS. 8A and 8B, the conductive pixels may be formed by an electrode pair including a top electrode, such as the first conductive pixel 810, and a bottom electrode, such as the second conductive pixel 808.

FIG. 8B illustrates an example where a portion of spacer fabric 812 is compressed and two of the conductive pixel sets are moved closer together. In this example, a portion of the flexible sensing strip 800 may detect forces while other portions of the flexible sensing strip 800 do not. This may permit, for example, multiple users to use a health monitoring device utilizing the flexible sensing strip 800 at the same time. For example, one user may compress one end of the flexible capacitive sensing mat 800 while another end is relatively uncompressed. An associated capacitive sense circuit may determine that only one user is present. If another user then compresses the other end of the flexible capacitive sensing mat 800, the capacitive sense circuit may determine that two users are present. In some embodiments, a compression width may be determined by detecting the width W of all compressed conductive pixel sets and by determining how many users (e.g., one user or two users) most likely corresponds to the detected width W.

In additional or alternative embodiments, the flexible sensing strip 800 may utilize multiple conductive pixel sets to detect a sleeping or resting position of a single user. For example, the compression of conductive pixels along a straight line (e.g., a line moving through two consecutive conductive pixel sets) may refer to a user lying in a rigid position (e.g., a position where a user's spine is substantially straight). In an additional example, a compression of conductive pixels along a curve may refer to a user lying in a curved position (e.g., a fetal position). The aforementioned posture detection may be performed periodically so as to track a user's posture throughout a period of time (e.g., a night). Processing electronics may be used to map the compressed pixels and to determine a user's posture.

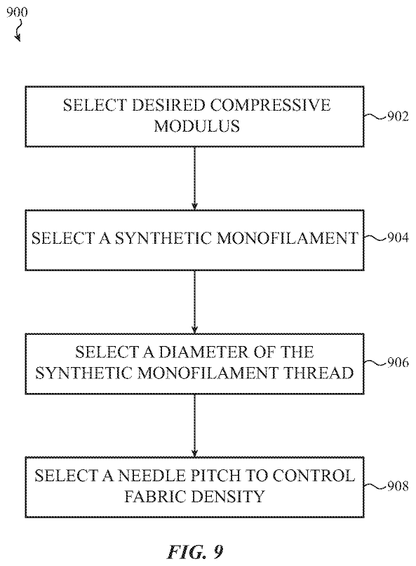

FIG. 9 depicts an example process 900 for determining the composition of a spacer fabric as used within a flexible capacitive sensing mat. As the resiliency and stiffness is largely dependent on the spacer fabric properties, the spacer fabric may be tunable to be suitable for a number of different application.

At operation 902, a desired compressive modulus of a spacer fabric may be determined. In some embodiments, the desired compressive modulus may be based on an application of the spacer fabric. For example, if the spacer fabric will be used in a health monitoring device as discussed herein, a compressive modulus may be desired to be less than commonly available mattresses. In another example, if the spacer fabric will be used in an article of clothing designed to be worn by a user, the compressive modulus may be set higher in response to anticipated dynamic movement.

As discussed herein, the compressive modulus of the spacer fabric may be between 5 kPa and 100 kPa or between 10 kPa and 20 kPa in instances where the spacer fabric will be used in a health monitoring device. When the spacer fabric will be used in a wearable application, such as in a watch or a blood pressure cuff, the compressive modulus may be higher, such as between 0.01 gigapascal (GPa) and 4 GPa. In some embodiments, the compressive modulus of a spacer fabric may be between 5 kPa and 100 kPa regardless of application.

At operation 904, a synthetic monofilament may be selected based on the desired compressive modulus. For example, where a relatively low compressive modulus is desired, a synthetic monofilament having a relatively low compressive modulus may be selected. Alternatively, where a relatively high compressive modulus is desired, a synthetic monofilament having a relatively high compressive modulus may be selected.

In some embodiments, a natural monofilament or a synthetic or natural multifilament may be selected instead of a synthetic monofilament.

At operation 906, a dial-cylinder distance and/or a loop length of a spacer fabric knitting machine may be determined. In some embodiments, a dial-cylinder distance may be selected to control a minimum compressed thickness and/or a compressive modulus. A dial-cylinder distance between 0 cm and 20 cm may be selected. Likewise, a loop length of the spacer fabric knitting machine may be selected to be between 5 cm and 25 cm.

At operation 908, a needle pitch may be selected to control a density of the spacer fabric. In embodiments where the spacer fabric is comprised of a monofilament, an angle of the monofilament with respect to an upper or lower surface may control the spacer fabric density. A spacer fabric with a relatively high density may be more difficult to compress and may fail to detect certain pressures. Similarly, a spacer fabric with a relatively low density may be easy to compress but may detect pressures that are undesirably small. By controlling the monofilament angle, a desired sensitivity may be achieved.

As described above, one aspect of the present technology is determining user presence on a mattress or padded furniture, pressure measurements, biological parameters, and so on. The present disclosure contemplates that in some instances this gathered data may include personal information data that uniquely identifies or can be used to contact or locate a specific person. Such personal information data can include sleep patterns, sleep time, location-based data, data or records relating to a user's health or level of fitness (e.g., vital signs measurements, medication information, exercise information), or any other identifying or personal information.

The present disclosure recognizes that the use of such personal information data, in the present technology, can be used to the benefit of users. For example, the personal information data can be used to provide sleep pattern recommendations and history that are tailored to and/or derived from the user. Further, other uses for personal information data that benefit the user are also contemplated by the present disclosure. For instance, health, sleep, and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities implement and consistently use privacy policies and practices that are generally recognized as meeting or exceeding industry or governmental requirements for ensuring personal information data is kept private and secure. Such policies should be easily accessible by users, and should be updated as the collection and/or use of data changes. Personal information from users should be collected for the legitimate and reasonable uses of the entity and should not be shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (“HIPAA”); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of determining spatial parameters, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, sleep patterns may be provided based on non-personal information data or a bare minimum amount of personal information, such as events or states of the device associated with a user, other non-personal information, or publicly available information.

Other examples and implementations are within the scope and spirit of the disclosure and appended claims. For example, flexible capacitive sensor may be used on wearable fabrics, in fabric scales, and in other pressure sensing/measuring systems. Also, as used herein, including in the claims, “or” as used in a list of items prefaced by “at least one of” indicates a disjunctive list such that, for example, a list of “at least one of A, B, or C” means A or B or C or AB or AC or BC or ABC (i.e., A and B and C). Further, the term “exemplary” does not mean that the described example is preferred or better than other examples.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A flexible mat comprising:
a first conductive layer;
an electromagnetic shield;
a second conductive layer disposed between the first conductive layer and the electromagnetic shield;
a spacer fabric disposed between the first conductive layer and the second conductive layer, the spacer fabric having a first thickness and configured to:
compress to at least a second thickness when an input is applied to the flexible mat, the second thickness less than the first thickness; and
revert to the first thickness when the input is no longer applied to the flexible mat; and
a capacitive sense circuit electrically coupled to the second conductive layer and configured to generate a presence detection signal when the spacer fabric layer compresses to the second thickness.

2. The flexible mat of claim 1, wherein the spacer fabric comprises:
a first fabric layer;
a second fabric layer; and
a synthetic monofilament layer disposed between the first fabric layer and the second fabric layer.

3. The flexible mat of claim 2, wherein:
the first fabric layer and the second fabric layer are formed from at least one of:
a spandex material;
a polyethylene terephthalate material;
a cotton; or
a wool; and
the synthetic monofilament layer has a compressive modulus between 10 kPa and 20 kPa.

4. The flexible mat of claim 2, wherein the synthetic monofilament layer has a cross-shaped pattern.

5. The flexible mat of claim 1, wherein:
a threshold pressure is 1.5 kPa; and
the capacitive sense circuit generates the presence detection signal when the input applies the threshold pressure, or any pressure greater than the threshold pressure, to the flexible mat.

6. The flexible mat of claim 1, wherein:
the first conductive layer is bonded to the spacer fabric by a first adhesive layer; and
the second conductive layer is bonded to the spacer fabric by a second adhesive layer.

7. The flexible mat of claim 1, further comprising a line stitch disposed at an end of the flexible mat and configured to couple the first conductive layer, the second conductive layer, and the spacer fabric.

8. An electronic device comprising:
a flexible housing defining an interior volume;
a flexible sensing strip positioned within the interior volume and comprising:
a first conductive layer;
a second conductive layer; and
a spacer fabric disposed between the first conductive layer and the second conductive layer, the spacer fabric configured to deform in response to an input; and
a capacitive sense circuit electrically coupled to the flexible sensing strip and configured to:
generate a detection signal when the flexible sensing strip deforms in response to the input; and
enable an operation of a biometric sensor once the detection signal is generated.

9. The electronic device of claim 8, wherein:
the first conductive layer comprises a first conductive thread;
the second conductive layer comprises a second conductive thread;
the first conductive thread is woven into a first surface of the spacer fabric; and
the second conductive thread is woven into a second surface of the spacer fabric, the second surface opposite the first surface.

10. The electronic device of claim 8, wherein the biometric sensor is at least one of:
the flexible sensing strip;
a ballistocardiograph sensor;
a piezoelectric sensor;
a heart-tracking monitor; or
a micro-electromechanical system device.

11. The electronic device of claim 10, wherein:
the first conductive layer and the second conductive layer define a plurality of electrode pairs; and
each electrode pair of the plurality of electrode pairs comprises a first electrode in the first conductive layer and a second electrode in the second conductive layer.

12. The electronic device of claim 11, wherein the capacitive sense circuit generates the detection signal when a distance between the upper electrode and the lower electrode of any one of the plurality of electrode pairs satisfies a threshold.

13. The electronic device of claim 8, wherein:
the flexible sensing strip extends across a width of a mattress; and
the input corresponds to a presence of a user on the mattress.

14. The electronic device of claim 13, wherein the spacer fabric has a first gap material modulus lower than a second gap material modulus of the mattress.

15. The electronic device of claim 13, wherein the flexible sensing strip is aligned with a chest of the user while the user lies on the mattress.

16. A flexible mat comprising:
a first conductive layer;
a second conductive layer; and
a spacer fabric disposed between the first conductive layer and the second conductive layer, the spacer fabric comprising:
a first fabric layer;
a second fabric layer; and
a synthetic monofilament layer disposed between the first fabric layer and the second fabric layer.

17. The flexible mat of claim 16, further comprising an electromagnetic shield configured to prevent electromagnetic signals from interfering with the first conductive layer and the second conductive layer; wherein the second conductive layer is disposed between the electromagnetic shield and the first conductive layer.

18. The flexible mat of claim 16, wherein the synthetic monofilament layer has a compressive modulus between 10 kPa and 20 kPa.

19. The flexible mat of claim 16, wherein the synthetic monofilament layer has an angle, with respect to the first conductive layer, greater than or equal to 45 degrees.

20. The flexible mat of claim 16, wherein a thickness of the flexible mat when fully compressed is between 0.3 mm and 3 mm.
